(19) United States
(12) Patent Application Publication
(10) Pub. No.: US 2017/0311812 A1
(43) Pub. Date: Nov. 2, 2017

(54) BATTERY THERMAL MASS

(71) Applicant: Cambridge Temperature Concepts Ltd, Cambridge, Cambridgeshire (GB)

(72) Inventor: Shamus Louis Godfrey HUSHEER, London (GB)

(21) Appl. No.: 15/521,413

(22) PCT Filed: Oct. 23, 2015
(86) PCT No.: PCT/GB2015/053188

§ 371 Date: Apr. 24, 2017

(30) Foreign Application Priority Data
Oct. 28, 2014 (GB) 1419190.2

(51) Int. Cl. A61B 5/01; G01K 7/42; G01K 13/00

(52) U.S. Cl. CPC A61B 5/01; G01K 13/002; G01K 7/427; A61B 2562/043; A61B 2562/0271; A61B 2560/0214; A61B 2503/40; A61B 2562/18; A61B 2562/14

(57) ABSTRACT

A device for measuring body temperature comprising: a first surface for thermal engagement with a body; a second surface substantially opposed to the first surface such that, in use when the first surface is engaged with a body, the second surface is exposed to a thermal environment of the body; first and second temperature sensors encapsulated within a first material; and a second material located between the first and second temperature sensors and intersecting a first axis passing substantially through the first and second sensors and the first and second surfaces; the device being configured such that the net thermal conductivity across the device is greatest along the first axis; and the second material having a volumetric heat capacity which substantially exceeds that of the first material.

### BACKGROUND OF THE INVENTION

This invention relates to a device for measuring the temperature of an animal or human body.

Sensors for measuring temperature are well known and include thermistors, thermocouples and semiconductor-based electronic sensors. If correctly calibrated, such sensors can provide an indication of the temperature of an object in the region from which the sensor takes its input. For example, a thermistor placed in direct contact with an object will give an indication of the temperature of that part of the object with which the sensor is in contact.

Often, an object does not have a uniform temperature and its measured temperature varies throughout its volume. For example, the temperature of an animal or human typically varies from its core body temperature to skin temperature. Skin temperature can vary considerably with environmental conditions and it is therefore the core body temperature which is typically more important for medical and diagnostic applications. However, it is not always possible or convenient to measure core body temperature directly by invasive means. It is preferable to make one or more measurements of an easily accessible part of the body (such as skin temperature) and estimate core body temperature from those measurements.

An example of a conventional device 12 for measuring the temperature of a body 11 is shown in FIG. 1. Temperature sensors 13 and 14 are arranged at different distances from the external surface 18 of body 11 in material 15, and are separated by a thermally-insulating barrier 16. The effect of thermally-insulating barrier 16 is to cause temperature sensors 13 and 14 to attain different equilibrium temperatures at different rates, such that a measurement of the temperature of body 11 can be estimated from the heat flow across the device between the first and second sensors.

Conventional devices measure the heat flow from the subject body into the device and require that the temperature sensors are accurately positioned so as to properly capture the flow of heat across the device and minimise the influence of environmental temperature changes. The accuracy of such devices is therefore heavily dependent on the accuracy of placement of the sensors of the device.

International patent publication WO 2010/023255 discloses an improved device for measuring body temperature which is configured such that the thermal conductivity of the device through the temperature sensors is greater than the thermal conductivity in other directions. This helps to establish a path for heat flow from the body through the device in a direction along an axis on which the temperature sensors are arranged and minimises the leakage of heat to the sensors from the lateral extremities of the device.

It is necessary to include various electronics in a device for measuring temperature. For example, in the case of a temperature data logger, the device would typically include the temperature sensors, a processor for acquiring temperature measurements and storing the measurements in a memory, and a battery for powering the processor. The thermal influence of these components on the heat flow through a device for measuring temperature can be significant, especially when the device is compact and the components consume a significant volume of the device. It is therefore important to package the components of the device so as to minimise their negative effects on the performance of the device.

### BRIEF SUMMARY OF THE INVENTION

According to a first aspect of the present invention there is provided a device for measuring body temperature comprising:
a first surface for thermal engagement with a body;
a second surface substantially opposed to the first surface such that, in use when the first surface is engaged with a body, the second surface is exposed to a thermal environment of the body;
first and second temperature sensors encapsulated within a first material; and
a second material located between the first and second temperature sensors and intersecting a first axis passing substantially through the first and second sensors and the first and second surfaces;
   the device being configured such that the net thermal conductivity across the device is greatest along the first axis; and
   the second material having a volumetric heat capacity which substantially exceeds that of the first material.

The first material may have a volumetric heat capacity of no more than 2000 kJ/m³K and the second material may have a volumetric heat capacity of at least 2000 kJ/m³K, the volumetric heat capacities of the first and second materials differing by at least 20%.

The second material may be a battery for powering the device.

The volumetric heat capacity of the second material may exceed the volumetric heat capacity of the first material by at least 20%, at least 30%, at least 40%, or at least 50%.

The second material extending substantially across the device in directions orthogonal to the first axis.

The device may further comprise a third temperature sensor encapsulated within the first material and located on the first axis between the first temperature sensor and the second material.

The first and third temperature sensors may be supported at a first PCB arranged substantially orthogonal to the first axis, the first PCB intersecting the first axis between the first and third temperature sensors.

The device may further comprise a fourth temperature sensor encapsulated within the first material and located on the first axis between the second temperature sensor and the second material.

The second and fourth temperature sensors may be supported at a second PCB arranged substantially orthogonal to the first axis, the second PCB intersecting the first axis between the second and fourth temperature sensors.

The net thermal conductivity across the device may be lowest in directions substantially perpendicular to the first axis.

The first material may have an anisotropic thermal conductivity, the first material being oriented such that its axis of greatest thermal conductivity is substantially aligned with the first axis.

The thermal conductivity of the first material may have an anisotropy ratio of at least 2.

The first material may be a thermally conductive polymer.

The third material may overlie the first material in regions of the device remote from the first axis, the third material not overlying the first or second surfaces and having a lower thermal conductivity than the first material.

The first material component may have, in the direction of the first axis, a greater thermal conductivity than the third material component by a factor of at least 4.

The first material may be substantially disc-shaped, the first axis being the axis of symmetry of the disc, and the third material being a ring-shaped annulus about the disc-shaped first material.

The second material may be substantially disc-shaped.

The first material may encapsulate the second material.

The thermal conductivity of the device may be substantially radially symmetric about the first axis.

The first surface may be adapted for engagement with the skin of a human or animal body.

The device may further comprise a processor configured to estimate a core body temperature of a subject human or animal from measurements of temperature acquired at the temperature sensors of the device.

According to a second aspect of the present invention there is provided a device for measuring body temperature comprising:
a first surface for thermal engagement with a body;
a second surface substantially opposed to the first surface such that, in use when the first surface is engaged with a body, the second surface is exposed to a thermal environment of the body; and
therebetween, a plurality of pairs of temperature sensors lying substantially along a first axis, the pairs of temperature sensors being encapsulated within a first material and each pair being separated by a thermal mass intersecting the first axis and having a volumetric heat capacity which substantially exceeds that of the first material;
   the device being configured such that the net thermal conductivity across the device is greatest along the first axis.

### BRIEF DESCRIPTION OF THE DRAWINGS

The present invention will now be described by way of example with reference to the accompanying drawings. In the drawings:

FIG. 1 shows a prior art device for measuring body temperature.

FIG. 2 is a schematic diagram of a device for measuring body temperature according to a first example.

FIG. 3 is a schematic diagram of a device for measuring body temperature according to a second example.

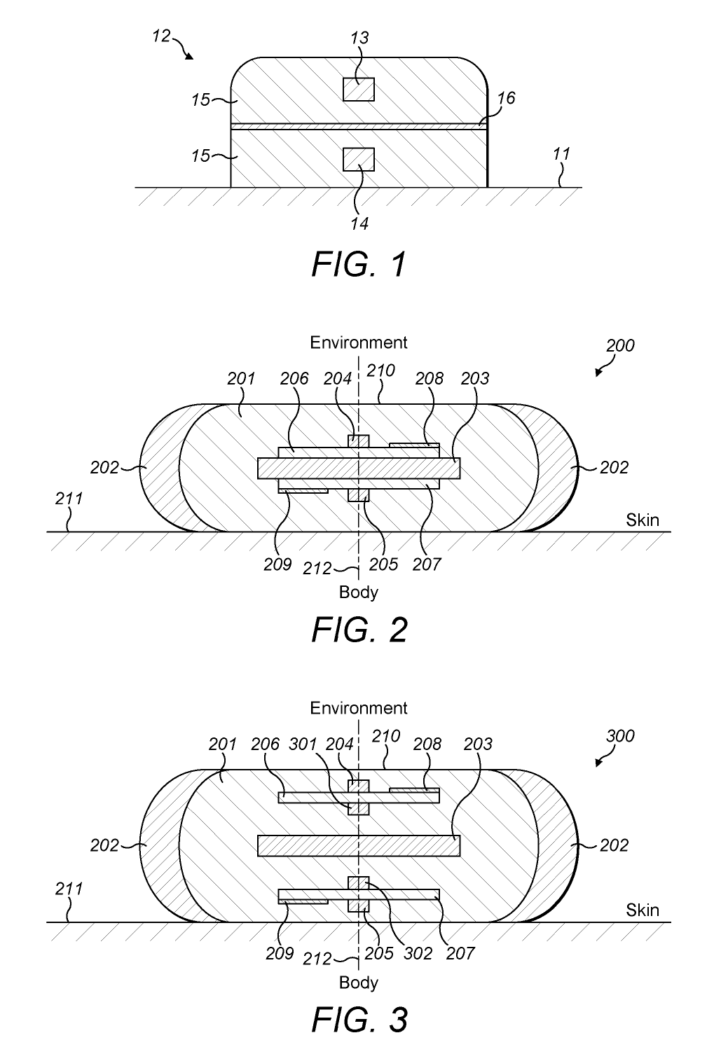

### DETAILED DESCRIPTION OF THE INVENTION

The following description is presented by way of example to enable any person skilled in the art to make and use the invention. The present invention is not limited to the embodiments described herein and various modifications to the disclosed embodiments will be readily apparent to those skilled in the art.

In thermal terms, the most significant electronic component in devices for measuring body temperature can often be the battery, especially for disposable devices which are not designed to be rechargeable and therefore include a relatively large battery. Previously this has presented a problem for such devices because the thermal characteristics of the battery can substantially alter the flow of heat through the device. This can lead to inaccurate estimates of heat flow.

The inventors have found that rather than negatively influencing measurements of heat flow at a device, through careful design the battery can be used to improve the performance of a device for measuring body temperature. More generally, the same benefits can be realised for all kinds of devices for measuring body temperature (e.g. including devices which include a very small battery or which are externally powered) through the advantageous arrangement of material properties described herein.

A measure of the heat flow from a body combined with a measure of the temperature at the surface of that body allows the calculation of an estimate of a temperature within the body, if one knows something of the thermal characteristics of the body. For example, the core body temperature (T_{core}) of a human or animal may be estimated from a first temperature T1 taken at a first point (such as at the skin) and a second temperature T2 measured at a second point (such as at the surface of the device remote from the body) related to the first point by a known thermal transfer function. For a device in which heat flow occurs in a substantially linear fashion past the sensors, these parameters allow the calculation of the heat flowing out of the skin in this region. Typically, for a given device an estimate of the core temperature from the measured temperatures T1 and T2 can be written as:

T_{core}=T1+A·(T2−T1) (1)

Parameter A would generally be empirically determined and depends on the thermal characteristics of the device (the thermal transfer function) and the body tissue. For example, parameter A can be established for equation (1) from measurements T1 and T2 acquired using the device, and accurate measurements of core body temperature, T_{core} (e.g. from an internal temperature probe). Including higher order terms in the equation can further improve the accuracy of this estimate. Measurements of other physiological temperatures can be performed in a similar manner. For example, a measurement of basal body temperature could be determined from an estimate of T_{core} taken when T_{core} is considered to be at its daily minimum for the human or animal subject.

Equation (1) represents a simple one-dimensional solution to the heat equation:

$$\frac{\partial u}{\partial t} = ∝ ( \frac{{\partial }^{2} u}{\partial {x}^{2}} ) \qquad ( 2 )$$

where u is temperature, x is distance, and α is a positive constant.

FIG. 2 is a schematic diagram of a device for measuring the temperature of a human or animal body. In this example, the device is adapted for attachment to the skin (or more generally, to the hide, fur, hair, feathers etc) of the body whose temperature is to be measured. The device comprises temperature measurement electronics encapsulated within a material 201. Material 201 may comprise one or more material components so as to provide the desired thermal characteristics described herein. Material 201 need not be homogeneous but it could be. In particular, the material 201 may comprise one or more coatings at its surfaces (e.g. surfaces 211 and 210). The electronics include temperature sensors 204 and 205 arranged along an axis 212 either side of a battery 203. Roughly speaking, sensor 205 is arranged to capture a measurement of the skin (or outer) temperature of the body, and sensor 204 is arranged to capture a temperature measurement indicative of the flow of heat out of the body, through the sensor and into the environment.

The temperature sensors encapsulated within material 201 lie entirely within the material or lie substantially within the material so as to expose part of the sensor for coupling to the body or environment.

The axis represents the direction of greatest thermal conductivity across the device as a whole (i.e. its ‘net’ thermal conductivity). This can be achieved in any suitable manner through particular material choices. For example, material 201 may itself have an anisotropic thermal conductivity such that its thermal conductivity in some directions is greater than in others. Through appropriate orientation of the material, axis 212 of greatest thermal conductivity can be defined.

Alternatively or additionally, axis 212 can be defined through the use of insulating material 202 in lateral regions of the device remote from the axis. By arranging that the thermal conductivity of the material 202 is lower than that of material 201, the direction of maximal thermal conductivity 212 can be defined through the temperature sensors. In FIG. 2, the insulating material forms an insulating annular ring around the disc shaped device. The insulating material could be a thermoplastic, such as polyvinyl chloride (PVC) or polyurethane (PU).

The axis of greatest thermal conductivity extends between a contact surface 211 of the device and an ambient surface 210. The contact surface thermally couples the device to the body whose temperature is to be measured. The contact surface may be provided with an adhesive to stick the device in place. Alternatively, the device may be held in place on a body by a strap, band or any other suitable means. The ambient surface is an opposing surface of the device which, when the device is worn by a human or animal user, is exposed to the thermal environment of the user. Depending on the configuration of the device and where it is worn, the environment of the device could be its thermal environment under clothing, fur, hair, feathers (as appropriate) and need not be exposed to the open air.

In preferred embodiments, material 201 comprises a polymer selected so as to provide an appropriate level of thermal conductivity along axis 212 relative to the thermal conductivity of the. A thermal conductivity of around 0.5 to 3 W/mK has been found to offer good performance. This can be achieved through the use of a thermally conductive polymer such as D8102 manufactured by Cool Polymers which has a thermal conductivity of 3 W/mK. Lower thermal conductivities can be achieved by blending the thermally conductive polymer with an insulating polymer.

In the example shown in FIG. 2, device 200 is substantially disc-shaped having greater extent parallel to its surfaces 210 and 211 than normal to those surfaces. For example, an appropriate diameter for the device as a skin sensor for the human body is approximately 20-30 mm. Suitable batteries for such a device include a CR2032 battery, or similar, with a metal (e.g. stainless steel) casing.

The temperature sensors 204 and 205 are arranged either side of battery 203 so that the battery provides an equivalent thermal inertia to each of the temperature sensors. This ensures that heat flow though the device along axis 212 can be accurately modelled by a one-dimensional heat flow equation, which simplifies the processing required to form an estimate of a core (or other physiological) temperature of the body from the temperature measurements acquired by sensors 204 and 205.

Providing a thermal mass such as battery 203 between the temperature sensors additionally helps to temporally isolate the sensors from one another. For example, if the environmental temperature changes suddenly, this change will be quickly reflected in the temperature measured by sensor 204, but will take longer to be reflected in the temperature measured by sensor 205 due to the thermal inertia presented by battery 203. The device can therefore provide information indicating whether a change in the body temperature measured by the device is truly a change in body temperature or is due to a change in environmental temperature. This information can be invaluable when trying to identify changes in body temperature from the data acquired by device 200. It is generally preferred that the thermal mass extends substantially across the device in a direction orthogonal to axis 212 since this provides good temporal isolation and supports the possibility of modelling heat flow through the device in one dimension (i.e. along axis 212). The thermal mass 203 further acts to smooth temperature fluctuations which is advantageous when measuring body temperature since rapid changes in temperature tend to be due to changes in environmental conditions whereas the device is directed to measuring the slower changes which occur in body temperature.

In FIG. 2, the temperature sensors are supported at PCBs 208 and 207 which are positioned between the battery and the respective temperature sensor. In this arrangement the PCBs could be considered to form part of the thermal mass provided between the sensors. In other examples, each temperature sensor could instead be located between its PCB and the battery. The PCBs would typically support one or more other components such as processor 208 (e.g. a low power system on a chip, or SoC) and a memory 209. In some examples, the processor could be configured to estimate a body temperature from the temperature measurements acquired at sensors 204 and 205—for example, in accordance with equation (1) above. The processor could be configured to store the estimated body temperature measurement and/or acquired temperature measurements at the memory. A transceiver and antenna (not shown) could also be provided to allow the acquired data to be transmitted to a receiver. The thermal masses of electronic components other than the battery are generally substantially less than that of the battery and can therefore be ignored for the purposes of heat flow calculations (e.g. for estimating body temperature).

The thermal mass provided between the sensors is chosen to have a greater volumetric heat capacity than material 201. In particular, good performance has been found using a volumetric heat capacity for the thermal mass of at least 2000 kJ/m³K (preferably at least 2500 kJ/m³K, more preferably at least 3000 kJ/m³K, and most preferably at least 3500 kJ/m³K), and a volumetric heat capacity for material 201 of no more than 2000 kJ/m³K (preferably no more than 1800 kJ/m³K, more preferably no more than 1650 kJ/m³K, and most preferably no more than 1500 kJ/m³K). It is however further important for the volumetric heat capacities to differ substantially. Good performance has been observed with volumetric heat capacities for the thermal mass and material 201 which differ by at least 20%, at least 30%, at least 40% and most preferably at least 50%.

In FIG. 2 the thermal mass is a battery. This provides the improved performance benefits described herein as well as representing an optimal packaging solution for small temperature sensing devices with relatively large batteries. In other examples, the thermal mass could be any material or aggregation of materials or components located between the temperature sensors on the axis of greatest thermal conductivity through the device. The composition of material 201 could be of any suitable form, including that of a single material, a mix of materials, or one or more discrete material components.

FIG. 3 is a schematic diagram showing a preferred device 300 and an improvement over the device 200 shown in FIG. 2. Device 300 includes two additional sensors, 301 and 302. These additional sensors define sensor pairs 301, 204 and 302, 205 lying either side of the thermal mass (in this case a battery) 203. Each of these sensor pairs can be used to determine a heat flow through the device between the sensors of the pair. Two different estimates of heat flow can therefore be identified: from the body into the thermal mass of the device, and from the thermal mass to the environment. Since further temperature data points indicative of the flow of heat through the device are provided by the sensors of device 300, the device offers improved accuracy. This is both because statistically a greater number of data points are available, and because separate models can be used for the heat flow either side of the thermal mass. In some examples, material 201 could have a different structure and/or composition either side of the thermal mass 203 so as to provide a different heat capacity in that respective region of the device and/or a different thermal conductivity.

The temperature sensors of each pair are preferably spatially separated along the axis 212 of the device such that the sensors measure at different points of the temperature gradient along axis 212. Sufficiently good performance can be achieved by mounting the temperature sensors on either side of a printed circuit board (PCB), which also provides a good and robust packaging solution. A PCB typically has a volumetric heat capacity of around 2000 kJ/m³K. Alternatively, the sensors could be separated by material 201 or any other suitable material, such as a thin layer of insulator.

Providing two pairs of temperature sensors either side of a thermal mass having the characteristics defined herein can offer very high performance and allow small changes in body temperature to be detected in a noisy thermal environment. The thermal mass allows the heat flow through one pair of temperature sensors to be calculated independently from the other pair. Even at a slow rate of temperature data capture (e.g. once per minute), this improves the ability to capture temporal data indicating whether a change in device temperature was due to a change in environmental conditions or a change in body temperature. For example, device 300 might be worn under the arm and arranged to capture data for the estimation of core or basal body temperature of a human user. If the device is worn and capturing data at night (e.g. in order to allow an estimate of basal body temperature to be formed), a large change in environmental temperature would be seen to occur if the user rolled over at night and lifted their arm to expose the device. The proposed device structure would allow temperature data to be captured which indicates that the resulting changes in temperature data were due to a sudden change in environmental temperature, and could potentially be corrected for (e.g. data from the temperature sensors could be processed according to a different heat flow model).

More generally, there could be two, three, four or more temperature sensors arranged such that at least one sensor lies either side of the thermal mass on the axis of greatest thermal conductivity through the device. There could be one or more batteries present in the device. There could be a plurality of sensor pairs, each pair being separated by a thermal mass (e.g. a battery) so as to provide improved resolution as to the flow of heat through the device and hence an improved estimate of body temperature.

The devices shown in FIGS. 2 and 3 may be manufactured by over-moulding the electronics (including the temperature sensors, PCBs and battery) with a polymer mix 201, and then over-moulding the resulting disc-shaped article with a thermally insulating polymer 202 predominantly in a ring laterally about the disc. Depending on the electrical conductivity of polymer 201, a thin electrically-insulating layer or film may be employed between the PCB, its electrical connections and polymer 201.

It is advantageous if the directions of lowest thermal conductivity across the devices 200 and 300 is substantially perpendicular to the axis of greatest thermal conductivity 212 so as to minimise the leakage of heat to the sensors from the lateral portions of the device.

Suitable materials having anisotropic thermal conductivity include thermally conductive polymers having substantially aligned polymer chains and a material matrix of electrically conductive components (such as metal fibres) aligned in a polymeric insulating base material. Material 201 may be selected so as to have an anisotropy ratio of at least 2, or more preferably at least 5: i.e. the thermal conductivity along the axis of greatest thermal conductivity is at least twice that along a substantially perpendicular axis of lowest thermal conductivity. In order to preserve the ability to model heat flow across the device in one dimension, it is further advantageous that the thermal conductivity in all directions perpendicular to the axis of greatest thermal conductivity is substantially the same (e.g. to within 20%). This can be expressed as the thermal conductivity of the device about axis 212 being rotationally symmetric.

The present disclosure is directed to a device for measuring the temperature of a human or animal body. More generally it can be applied to the measurement of temperature of other bodies operating in a similar temperature range (e.g. between 0 and 100 degrees Centigrade).

Some non-limiting examples of uses to which data derived from the sensor may be put are assisting natural conception, natural contraception, artificial insemination, in-vitro fertilisation (IVF), detecting or predicting ovulation, skin care, assisting post-operative recovery and diagnosis, assisting weight management, baby monitoring, monitoring sports performance, monitoring performance in extreme environments, tamper evidence, wearer tracking, in-hospital monitoring of bodily functions, assisting fitness, health, wellbeing or activity management, and detection, diagnosis, treatment, management or background monitoring for of any of the following conditions: chronic obstructive pulmonary disease (COPD), cystic fibrosis (CF), diabetes, hypoglycaemia, sleep disturbance, sleep apnoea, chronic pain, infection (e.g. by bacterial, viral, prion, protozoal, fungal or parasitic agents), sepsis, polycystic ovary syndrome (PCOS), menopause, asthma, insomnia, schizophrenia, coronary heart disease, narcolepsy, restless legs syndrome, rheumatoid arthritis, inflammatory bowel disease (IBD), lupus, periodic fever syndromes and cancers such as lymphoma, leukaemia and renal cancer. The sensor may be applied to humans or animals.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A device for measuring body temperature comprising:
a first surface for thermal engagement with a body;
a second surface substantially opposed to the first surface such that, in use when the first surface is engaged with a body, the second surface is exposed to a thermal environment of the body;
first and second temperature sensors encapsulated within a first material; and
a second material located between the first and second temperature sensors and intersecting a first axis passing substantially through the first and second sensors and the first and second surfaces;
the device being configured such that the net thermal conductivity across the device is greatest along the first axis; and
the second material having a volumetric heat capacity which substantially exceeds that of the first material.

2. A device as claimed in claim 1, the first material having a volumetric heat capacity of no more than 2000 kJ/m³K and the second material having a volumetric heat capacity of at least 2000 kJ/m³K, the volumetric heat capacities of the first and second materials differing by at least 20%.

3. A device as claimed in claim 1, the second material being a battery for powering the device.

4. A device as claimed in claim 1, the volumetric heat capacity of the second material exceeding the volumetric heat capacity of the first material by at least 30%, at least 40%, or at least 50%.

5. A device as claimed in claim 1, the second material extending substantially across the device in directions orthogonal to the first axis.

6. A device as claimed in claim 1, further comprising a third temperature sensor encapsulated within the first material and located on the first axis between the first temperature sensor and the second material.

7. A device as claimed in claim 6, the first and third temperature sensors being supported at a first PCB arranged substantially orthogonal to the first axis, the first PCB intersecting the first axis between the first and third temperature sensors.

8. A device as claimed in claim 1, further comprising a fourth temperature sensor encapsulated within the first material and located on the first axis between the second temperature sensor and the second material.

9. A device as claimed in claim 8, the second and fourth temperature sensors being supported at a second PCB arranged substantially orthogonal to the first axis, the second PCB intersecting the first axis between the second and fourth temperature sensors.

10. A device as claimed in claim 1, the net thermal conductivity across the device being lowest in directions substantially perpendicular to the first axis.

11. A device as claimed in claim 1, the first material having an anisotropic thermal conductivity, the first material being oriented such that its axis of greatest thermal conductivity is substantially aligned with the first axis.

12. (canceled)

13. A device as claimed in claim 1, the first material being a thermally conductive polymer.

14. A device as claimed in claim 1, further comprising a third material overlying the first material in regions of the device remote from the first axis, the third material not overlying the first or second surfaces and having a lower thermal conductivity than the first material.

15. A device as claimed in claim 14, the first material component having, in the direction of the first axis, a greater thermal conductivity than the third material component by a factor of at least 4.

16. A device as claimed in claim 14, the first material being substantially disc-shaped, the first axis being the axis of symmetry of the disc, and the third material being a ring-shaped annulus about the disc-shaped first material.

17. A device as claimed in claim 1, the second material being substantially disc-shaped.

18. A device as claimed in claim 1, the first material encapsulating the second material.

19. A device as claimed in claim 1, the thermal conductivity of the device being substantially radially symmetric about the first axis.

20. (canceled)

21. A device as claimed in claim 1, further comprising a processor configured to estimate a core body temperature of a subject human or animal from measurements of temperature acquired at the temperature sensors of the device.

22. A device for measuring body temperature comprising:
a first surface for thermal engagement with a body;
a second surface substantially opposed to the first surface such that, in use when the first surface is engaged with a body, the second surface is exposed to a thermal environment of the body; and
therebetween, a plurality of pairs of temperature sensors lying substantially along a first axis, the pairs of temperature sensors being encapsulated within a first material and each pair being separated by a thermal mass intersecting the first axis and having a volumetric heat capacity which substantially exceeds that of the first material;
the device being configured such that the net thermal conductivity across the device is greatest along the first axis.

23. (canceled)
